(19) United States
(12) Patent Application Publication
(10) Pub. No.: US 2010/0063408 A1
(43) Pub. Date: Mar. 11, 2010

(54) Breathalyzer having pivoted mouthpiece

(76) Inventors: Keith H. Nothacker, San Francisco, CA (US); Michael J. Strasser, San Francisco, CA (US); Ralf Groene, Kirkland, WA (US); Greg Cerny, Palo Alto, CA (US)

Correspondence Address: William C. Milks, III, RUSSO & HALE LLP, 401 Florence Street, Palo Alto, CA, 94301, US

(21) Appl. No.: 12/283,572

(22) Filed: Sep. 11, 2008

(51) Int. Cl. A61B 5/00

(52) U.S. Cl. 600/532

(57) ABSTRACT

A breathalyzer includes a housing and incorporates a mouthpiece pivotally mounted in the housing and movable between a first, retracted position for reducing the exposure of the mouthpiece to damage and contaminants and a second, extended position for receiving exhaled breath of a person.

### BACKGROUND OF THE INVENTION

1. Field of the Invention

The present invention relates to measurement devices and, more particularly, to breathalyzers for measuring one or more substances present in the exhaled breath of a person, for example, for measuring the blood alcohol content of the person. Specifically, one preferred embodiment of the present invention provides a breathalyzer comprising a pivotally mounted mouthpiece that is movable between a retracted position and an extended position for deployment to receive the exhaled breath of a person, in which the deployment of the mouthpiece is preferably coordinated with activating the breathalyzer measurement circuitry, and the circuitry is deactivated when the mouthpiece is retracted.

2. Description of the Prior Art

By way of background, breathalyzers are employed by law enforcement officers and hospitals and more recently by businesses and individuals to test for blood alcohol content, for example. It is well-known that motor vehicle laws prohibit driving, and job safety requirements preclude operating machinery, while under the influence of alcohol. Various breathalyzer configurations are known having fixed mouthpieces into which a person breathes to test for blood alcohol content based on a deep lung air sample. The fixed mouthpieces are susceptible to breakage and exposed to environmental contaminants, for example, airborne dust and bacteria, which may accumulate on the mouthpiece, resulting in unsanitary and unhealthy conditions.

In order to address the potential health problem, various known breathalyzers comprise disposable mouthpieces. For example, Wolf, U.S. Pat. No. 5,291,898 discloses a breath alcohol device comprising two halves of a housing that define a mouthpiece-receiving collar with an ejector slot running axially along its bottom, stopping short of the outer end. A disposable mouthpiece is inserted through the port collar. As it is pushed inwardly, a flange engages a finger of an actuator-ejector. Further movement of the mouthpiece toward a mouthpiece receiver rocks a barrel around a bearing post, causing a drive lug to move a slide toward a cover. By the time the mouthpiece is seated in the mouthpiece receiver, the slide has moved to the position at which the cover is closed, a finger is flexed against its natural bias, and a latch blade is snapped into place in a keeper notch. The rocking of the barrel also brings a finger into position on the outer side of the flange, so that the flange is caged between the two fingers and the mouthpiece cannot be withdrawn. The rotation of the barrel also moves a cam to a position at which a switch lever is moved, by the bias of an operator, away from a switch, which permits the operator to close the circuit energizing the circuitry of the device. At any time after the readout appears, an ejector button can be depressed, and the bias of a spring not only causes the slide to move away from the cover, but causes the finger to eject the mouthpiece forcibly, so that the mouthpiece is jettisoned into a refuse container or onto the ground without the need for an officer administering a measurement to touch the used mouthpiece. The structure disclosed in the Wolf patent is complex and costly to construct, and the cost is also increased by requiring disposable mouthpieces. The disposable mouthpiece when mounted in the port collar also extends from the breathalyzer in a similar manner to breathalyzers having a fixed mouthpiece and is therefore susceptible to breakage.

The problem of providing a breathalyzer that has a mouthpiece that is convenient to use and has a lower risk of breakage continues to exist. It is an object of the present invention to provide a breathalyzer whose mouthpiece is less susceptible to breakage. It is also an objective of the present invention to provide such a breathalyzer which is effective to reduce the additional problem of contamination in a cost-effective manner. Various embodiments of the present invention provide a solution to the breakage and contamination problems encountered by breathalyzers having fixed or disposable mouthpieces.

### SUMMARY OF THE INVENTION

In accordance with one aspect of the present invention, a breathalyzer is provided having a pivotally mounted mouthpiece that deploys when a release mechanism, for example, a slide button, is actuated to configure the mouthpiece for receiving an air sample. The mouthpiece is preferably spring-loaded to effect pivoted movement of the mouthpiece when the release mechanism is actuated. The mouthpiece is movable between a first, retracted position for safeguarding the mouthpiece and a second, extended position for receiving the exhaled breath of a person. In accordance with another aspect of the present invention, the deployment of the mouthpiece to the extended position is preferably coordinated with activating the breathalyzer measurement circuitry, and retraction of the mouthpiece deactivates the circuitry.

Accordingly, in accordance with the various embodiments of the present invention, the mouthpiece retracts for safe storage free from dirt and germs. The mouthpiece is spring-loaded and is released to the operative position by actuating a release mechanism, such as a slide button. When the slide button is actuated, the mouthpiece swings out, and the breathalyzer measurement circuitry comprising blood alcohol sensor electronics is also turned on to begin a warm-up sequence in preparation for a blood alcohol test. When actuated, the mouthpiece snaps out automatically, provides a closed entryway for airflow, and initiates the testing sequence. The mouthpiece retracts after use for safe storage to protect the mouthpiece from dirt and damage, and makes it more convenient for storage and transport. In accordance with another embodiment of the present invention, to promote hygiene, the mouthpiece may additionally comprise a removable cover that may be sterilized or, alternatively, a disposable cover for the mouthpiece that may be selectively slid on and off the mouthpiece.

The foregoing and other objects, features, and advantages of the present invention will become more readily apparent from the following detailed description of various embodiments of the present invention, which proceeds with reference to the accompanying drawing.

### BRIEF DESCRIPTION OF THE DRAWING

The various embodiments of the present invention will be described in conjunction with the accompanying figures of the drawing to facilitate an understanding of the present invention. In the figures, like reference numerals refer to like elements. In the drawing:

FIG. 1A is an isometric view of one embodiment of the breathalyzer in accordance with the present invention illustrating a mouthpiece in a retracted position for storage and/or transport of the breathalyzer.

FIG. 1B is an isometric view of the embodiment of the breathalyzer in accordance with the present invention shown in FIG. 1A illustrating the mouthpiece in an extended position for receiving the exhaled breath of a person who blows into the mouthpiece.

FIG. 2 is an exploded view of one preferred embodiment of the breathalyzer in accordance with the present invention shown in FIGS. 1A and 1B.

FIG. 3 is a detailed view of the tube assembly shown in FIG. 2.

FIG. 4A is a cross-sectional view of the breathalyzer shown in FIG. 1A with the mouthpiece in the retracted position.

FIG. 4B is a cross-sectional view of the breathalyzer shown in FIG. 1B with the mouthpiece in the extended position.

### DETAILED DESCRIPTION OF THE PREFERRED EMBODIMENTS

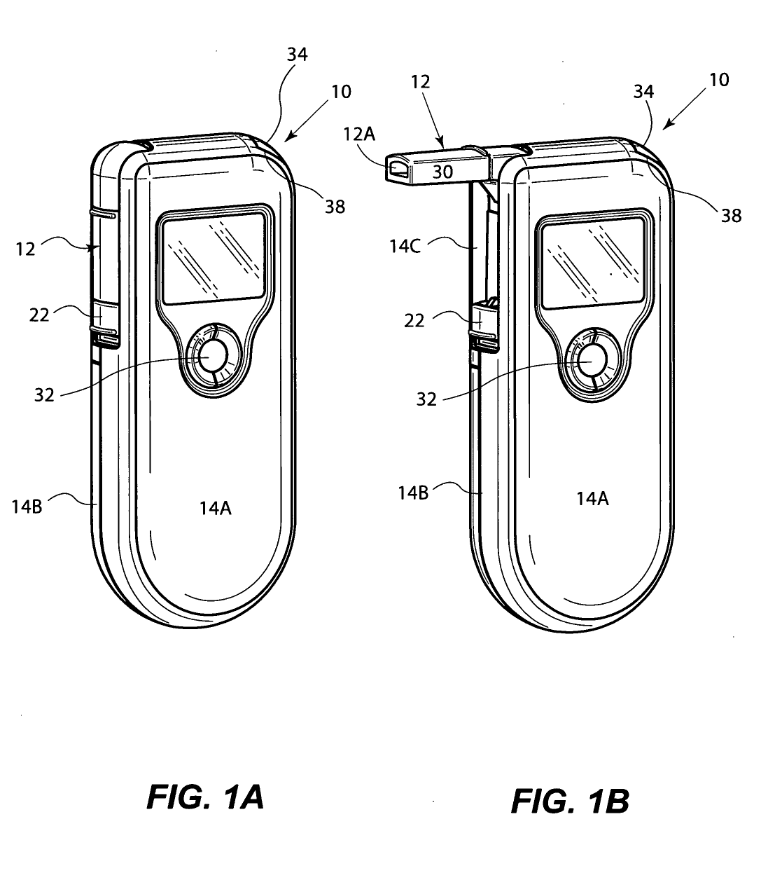

FIG. 1A is an isometric view of one embodiment of a breathalyzer 10 in accordance with the present invention illustrating a mouthpiece 12 in a retracted position for storage and/or transport of the breathalyzer. FIG. 1B is an isometric view of the embodiment of the breathalyzer 10 shown in FIG. 1A illustrating the mouthpiece 12 in an extended position in which the mouthpiece is deployed for receiving the exhaled breath of a person who blows into the mouthpiece.

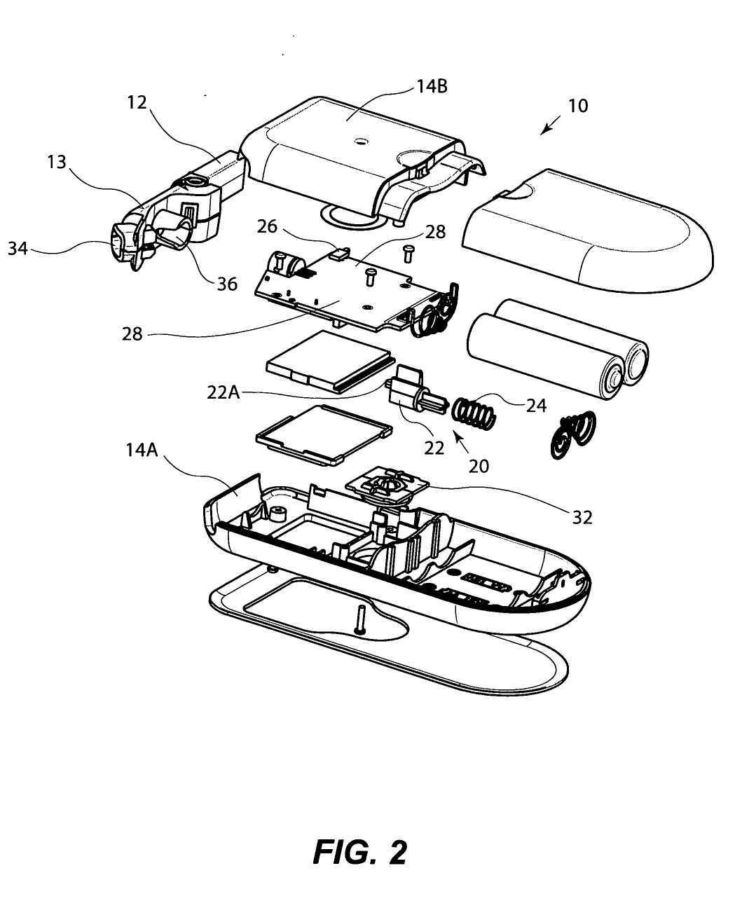

Considered in more detail, FIG. 2 is an exploded view of a preferred embodiment of the breathalyzer 10 in accordance with the present invention showing a spring-loaded pivotally mounted mouthpiece 12. The breathalyzer 10 comprises the mouthpiece 12 pivotally mounted in a tube assembly 13 which is in turn mounted in a housing comprised of a front housing member 14A and a back housing member 14B.

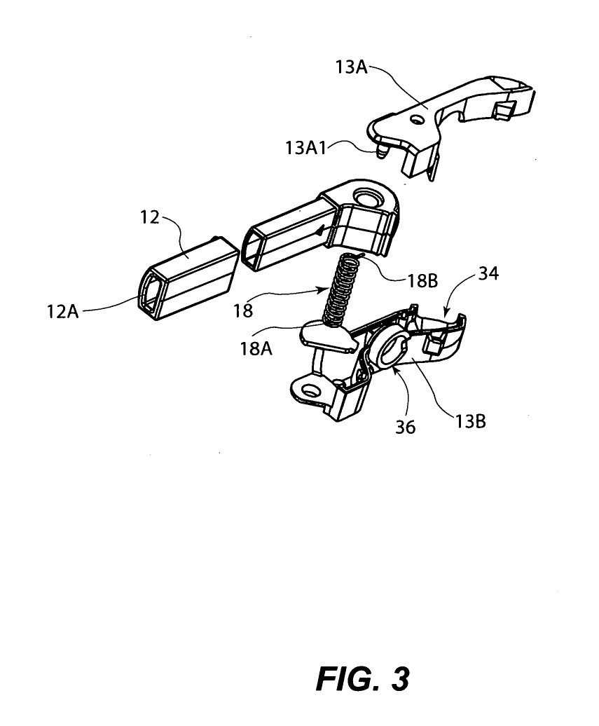

As shown in FIG. 3, the tube assembly 13 preferably comprises a front member 13A having a post 13A1 and also comprises a back member 13B. The mouthpiece 12 is pivotally mounted on the post 13A1 and is captured between the front member 13A and the back member 13B of the tube assembly 13.

Preferably, a torsional force is applied to the mouthpiece 12 to induce pivotal movement of the mouthpiece. Accordingly, a torsion spring 18 is provided having a first end 18A secured to the mouthpiece 12, and a second end 18B secured to the front member 13A of the tube assembly 13, which is in turn mounted within the housing 14A, 14B with the mouthpiece in the extended position, as shown in FIG. 1B. Accordingly, when the mouthpiece 12 is retracted, the torsion spring 18 becomes more tightly coiled and is thus biased to pivotally move the mouthpiece 12 from the retracted position shown on FIG. 1A to the extended position shown in FIG. 1B in which the mouthpiece is deployed for receiving the exhaled breath of a person who blows into the mouthpiece.

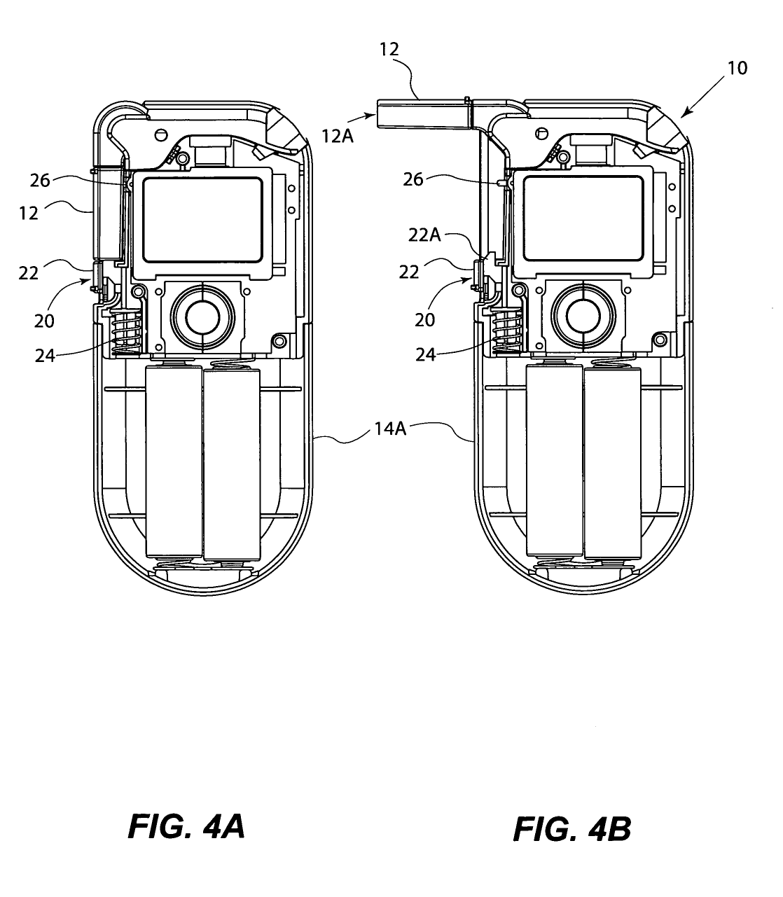

Referring again to FIG. 2, the breathalyzer 10 also preferably comprises a release mechanism 20 to selectively retain the mouthpiece 12 in the retracted position and enable the mouthpiece to be deployed to the extended position, as shown in FIGS. 1A and 1B, respectively. As shown in FIGS. 2, 4A, and 4B, the release mechanism 20 preferably comprises a slide button 22 that is slidably mounted between the front housing member 14A and back housing member 14B. The slide button 22 comprises a protrusion 22A that is biased toward the mouthpiece 12 by a slide button spring 24 into an opening 12A of the mouthpiece to retain the mouthpiece in the retracted position shown in FIGS. 1A and 4A. For example, the slide button spring 24 may be a conventional steel compression spring. Alternatively, in accordance with a contemplated alternative embodiment of the present invention, the slide button spring 24 may be a plastic leaf spring or other means to mechanically bias the slide button 22 into the opening 12A of the mouthpiece 12. It is also contemplated in accordance with other alternative embodiments of the present invention that the release mechanism 20 may be another type of release button such as a pushbutton release instead of a side button release as will be understood by persons skilled in the art.

When the release mechanism 20 such as the slide button 22 is manually actuated to compress the slide button spring 24, the protrusion 22A is withdrawn from the opening 12A of the mouthpiece 12, and the mouthpiece pivots from the retracted, closed position shown in FIGS. 1A and 4A, in which the mouthpiece is disposed within a cavity 14C formed within and parallel to the side of the housing 14A, 14B, and is deployed to the extended, open position approximately perpendicular to the side of the housing, as shown in FIGS. 1B and 4B. When the mouthpiece 12 deploys to the extended position, a switch 26 on a blood alcohol sensor electronics printed circuit board 28 is actuated to activate blood alcohol sensor electronics comprising the breathalyzer measurement circuitry.

After use, the mouthpiece 12 is manually pivoted toward the side of the housing 14A, 14B to the retracted position. As the mouthpiece 12 engages the protrusion 22A, the slide button 22 is forced away from the opening 12A of the mouthpiece until the protrusion clears the lip of the mouthpiece, at which time the slide button spring 24 biases the protrusion of the slide button into the opening of the mouthpiece to retain the mouthpiece in the retracted position for safe storage to protect the mouthpiece from dirt and damage.

Accordingly, in accordance with the various embodiments of the present invention, the mouthpiece 12 retracts for safe storage free from contaminants and potential breakage. The mouthpiece 12 is spring-loaded and is released to the operative position shown in FIGS. 1B and 4B by actuating the release mechanism 20 such as the slide button 22. When the slide button 22 is actuated, the mouthpiece 12 swings outwardly, and the blood alcohol electronics are also turned on to begin a warm-up sequence in preparation for a blood alcohol test. When actuated, the mouthpiece 12 snaps out automatically, provides a closed entryway for airflow, and initiates the testing sequence. The mouthpiece 12 retracts after use as shown in FIGS. 1A and 4A for safe storage to protect the mouthpiece from dirt and damage, and makes the breathalyzer 10 more convenient for storage and transport.

In accordance with another embodiment of the present invention, the mouthpiece 12 is adapted to accommodate a cover 30, as shown in FIG. 1B. For example, the cover 30 may be a sleeve such as a clear plastic sleeve, which is slid onto the mouthpiece 12. In accordance with one embodiment of the present invention, the cover 30 is reusable and thus is removable for cleaning and/or sterilization. Alternatively, in accordance with a contemplated alternative embodiment of the present invention, the cover 30 is disposable and may be a thin elastic sleeve that is temporarily fitted onto the mouthpiece 12 during use of the breathalyzer 10 and subsequently peeled off the mouthpiece and discarded, such that only the cover and not the entire mouthpiece is disposed of after use in contrast to the prior art as disclosed in the aforementioned Wolf patent, for example, in which the entire mouthpiece is discarded, thereby reducing the cost associated with use of the breathalyzer.

Preferably, as shown in FIGS. 1A, 1B, and 2, the breathalyzer 10 further comprises a switch assembly 32 coupled to the blood alcohol sensor electronics printed circuit board 28. When the mouthpiece 12 is deployed in the extended position shown in FIGS. 1B and 4B, the switch assembly 32 is enabled and may be actuated by a person to initiate a blood alcohol measurement without having to first move the mouthpiece to the retracted position shown in FIGS. 1A and 4A and again deploy the mouthpiece to the extended position shown in FIGS. 1B and 4B so as to operate the switch 26 to cause another measurement to be initiated.

In accordance with another preferred embodiment of the present invention, the breathalyzer 10 may additionally comprise an exhaust port structure 34, as shown in FIGS. 1A, 1B, 2, and 3. The exhaust port structure 34 enables the exhaled breath of a person, that enters through the mouthpiece 12, to be exhausted from the breathalyzer 10. At least a portion of the exhaled breath is directed to the blood alcohol sensor electronics printed circuit board 28 through a sample port 36.

Additionally, the exhaust port structure 34 may be employed as an inlet for the exhaled breath of a person instead of requiring the person to use the mouthpiece 12. Accordingly, while a less accurate blood alcohol content measurement may result, a person may simply blow into the exhaust port structure 34, such that at least a portion of the exhaled breath is directed to the blood alcohol sensor electronics printed circuit board 28 through the sample port 36. Preferably, the switch assembly 32 is enabled and may be actuated by the person to initiate a blood alcohol content measurement employing the exhaust port structure 34 without having to first move the mouthpiece 12 to the extended position shown in FIGS. 1B and 4B so as to operate the switch 26 to cause the measurement to be initiated. The person can therefore be tested by blowing into the exhaust port structure 34 without deploying the mouthpiece 12 to the extended position shown in FIGS. 1B and 4B. Preferably, the exhaust port structure 34 further comprises a lighted collar 38 such as a clear plastic ring and an LED light source for illuminating the ring to indicate that the breathalyzer 10 is enabled to receive exhaled breath through the exhaust port structure when the mouthpiece 12 is in the retracted position shown in FIGS. 1A and 4A, as well as to provide a target at which to blow. If the mouthpiece 12 is deployed to the extended position shown in FIGS. 1B and 4B, preferably, the light collar 38 is not illuminated to indicate that a blood alcohol content measurement may be performed by blowing into the exhaust port structure 34, and a person must blow into the mouthpiece 12 to obtain a measurement.

While the foregoing description has been with reference to particular embodiments and contemplated alternative embodiments of the present invention, it will be appreciated by those skilled in the art that changes in these embodiments may be made without departing from the principles and spirit of the invention. Accordingly, the scope of the present invention can only be ascertained with reference to the appended claims.

What is claimed is:

1. A breathalyzer comprising:
a housing; and
a mouthpiece pivotally mounted in the housing and movable between a first, retracted position for safeguarding the mouthpiece and a second, extended position for receiving exhaled breath of a person.

2. The breathalyzer of claim 1, further comprising a release mechanism actuated to configure the mouthpiece for receiving a breath sample.

3. The breathalyzer of claim 2 wherein the release mechanism comprises a slide button.

4. The breathalyzer of claim 2, further comprising a spring coupled to the mouthpiece to effect pivotal movement of the mouthpiece when the release mechanism is actuated.

5. The breathalyzer of claim 1, further comprising blood alcohol sensor electronics and a switch actuated by pivotal movement of the mouthpiece to activate the blood alcohol sensor electronics.

6. The breathalyzer of claim 5 wherein the switch is actuated upon retraction of the mouthpiece to deactivate the blood alcohol sensor electronics.

7. The breathalyzer of claim 1, further comprising a removable cover that may be cleaned or sterilized or a disposable cover for the mouthpiece, that may be selectively slid on and off the mouthpiece.

8. The breathalyzer of claim 1, further comprising an exhaust port structure to enable the exhaled breath of a person, that enters through the mouthpiece, to be exhausted from the breathalyzer.

9. The breathalyzer of claim 5, further comprising an exhaust port structure to enable the exhaled breath of a person, that enters through the mouthpiece, to be exhausted from the breathalyzer.

10. The breathalyzer of claim 9, further comprising a switch assembly actuated by the person to initiate a blood alcohol content measurement employing the exhaust port structure without having to first move the mouthpiece to the extended position so as to operate the switch to cause the measurement to be initiated.

11. The breathalyzer of claim 10 wherein the exhaust port structure further comprises a lighted collar that is activated when the mouthpiece is retracted and the switch assembly is actuated.
